(19) United States
(12) Patent Application Publication
(10) Pub. No.: US 2005/0221279 A1
(43) Pub. Date: Oct. 6, 2005

(54) Method for creating chemical sensors using contact-based microdispensing technology

(75) Inventors: J. Chance Carter, Livermore, CA (US); Billy W. Colston JR., San Ramon, CA (US); Steve B. Brown, Livermore, CA (US); Thomas S. Wilson, San Leandro, CA (US); Ujwal S. Setlur, Livermore, CA (US); Christine N. Paulson, Fremont, CA (US); Jane P. Bearinger, Livermore, CA (US)

Correspondence Address: Michael C. Staggs;Attorney for Applicants, Lawrence Livermore National Laboratory, P.O. Box 808, L-703, Livermore, CA, 94551, US

(73) Assignee: The Regents of the University of California

(21) Appl. No.: 11/099,274

(22) Filed: Apr. 4, 2005

Related U.S. Application Data
(60) Provisional application No. 60/559,834, filed on Apr. 5, 2004.

(51) Int. Cl. C12Q 1/00

(52) U.S. Cl. 435/4

(57) ABSTRACT

Contact based rigid pin tool technology is utilized to print one or more indicator chemistries on an optical array or a disposable sheath configured on such arrays. Each indicator chemistry contains predetermined material, such as, light energy absorbing dye(s), optically responsive particles, etc., whose optical characteristics change in response to the target ligand or analyte. By spectrally monitoring such changes using fluorescence and/or absorption spectroscopy, detection and/or quantitation of the target ligand or analyte can be obtained.

### CROSS-REFERENCE TO RELATED APPLICATIONS

This application claims the benefit of U.S. Provisional Application No. 60/559,834, filed Apr. 5, 2004, entitled “Method for Creating Chemical Sensors Using Contact-Based Microdispensing Technology”, which is incorporated herein by this reference.

The United States Government has rights in this invention pursuant to Contract No. W-7405-ENG-48 between the United States Department of Energy and the University of California for the operation of Lawrence Livermore National Laboratory.

### BACKGROUND OF THE INVENTION

1. Field of Endeavor

The present invention relates to chemical sensors, and more particularly to chemical sensors for detecting and/or analyzing at least one ligand or analyte of interest in a fluid or airborne medium utilizing a contact-based tool technology.

2. State of Technology

In the mid 1970's researchers began investigating the possibility of using optical fibers in sensing applications for measuring analytes remotely, in real-time and in-situ. Advances in such research led to the development of chemically immobilized, indicator-based fiber optic chemical sensors. Background for such information can be found in: “PCO₂-optode-PO₂-optode—new probe for measurement of PCO₂ or PO₂ in fluids and gases”, Z. Naturforsch, Section C, 30 (7-8): 532-533 (1975), D. W. Lubbers, N. Opitz; “Nano-encapsulated fluorescence indicator molecules measuring pH and PO₂ down to submicroscopical regions on basis of optode-principle”, Z. Naturforsch, Section C, 32 (1-2): 133-134 (1977) by D. W. Lubbers, N. Opitz, P. P. Speiser, H. J. Bisson, Adv. Exp. Med Biol. 94, 99 (1977), N. Opitz, H. Weigelt, T. Barankay, D. W. Lubbers; “Continuous Trancutaneous Blood Gas Monitoring, Birth Defects,” by D. W. Lubbers, F. Hannebauer, N. Opitz, Eds A. Huch, R. Huch, J. F. Lucey,. (Liss, New York, 1979), pp. 123-126; and “Optical fluorescence sensors for continuous measurement of chemical concentrations in biological-systems”, by D. W. Lubbers, N. Opitz, Sensors and Actuators, 4 (4): 641-654 (1983).

Over the past 25 years, intensive research has continued in the area of optical fiber-based chemical sensors with applications including process control, environmental, occupational safety, quality control, and biomedical. Background information on such sensors and applications can be found in: “CRC Critical Reviews” Anal. Chem. 19, 135 (1988) by W. R. Seitz et al.; Anal. Chem., 337, 522 (1990), by O. S. Wolfbeis, Fres. J.; and in ACS Symposium Series 403: 252 (1989) by D. R. Walt et al.

Typically, such fiber-based sensors include an indicator chemistry attached to the end of an optical fiber where the indicator chemistry exhibits certain optical characteristics (i.e. fluorescence intensity, fluorescence lifetime, or absorption) that change in response to the presence of a target analyte. In the case of a fluorescence-based indicator chemistry attached to the end of a fiber, light of a suitable wavelength is used to illuminate the attached indicator, resulting in a portion of that light being absorbed and subsequently re-emitted in the form of fluorescence (i.e. intensity, lifetime, etc). This process is monitored via photosensitive detectors (e.g. charge-coupled devices, photomultiplier tubes, photodiodes, etc.) and the resulting signal used to make qualitative and/or quantitative determinations concerning the target analyte.

Traditional methods for fabricating chemically immobilized, indicator-based optical fiber sensors involve attachment of the substrate by direct physical attachment, dip coating or photopolymerization methods. Background information for such methods can be found in: “A fiber optic pH probe for physiological use”, by J. I. Peterson, S. R. Goldstein, R. V. Fitzgerald, D. K. Buckhold, Anal. Chem., 52, 864 (1980); “pH sensor based on immobilized fluoresceinamine”, by L. A. Saari, W. R. Seitz, Anal. Chem. 54, 821 (1982); “A fluorescence sensor for quantifying pH in the range from 6.5 to 8.5”, by Z. Zhujun, W. R. Seitz, Anal. Chim. Acta 160, 47 (1984); “Optical fluorescence and its application to an intravascular blood gas monitoring system,” by J. L. Gehrich, D. W. Lubbers, N. Opitz, D. R. Hansmann, W. W. Miller, K. K. Tusa, and M. Yafuso, IEEE Trans. Biomed. Eng., 33, 117 (1986); “A fiber optic pH sensor using base catalyzed organo-silica sol-gel,” by D. A. Nivens, Y. Zang, S. M. Angel, Anal. Chem. Acta 376, 235 (1998); “Multilayer sol-gel membranes for optical sensing applications: single layer pH and dual layer CO₂ and NH₃ sensors,” by D. A. Nivens, M. V. Schiza, and S. M. Angel, Talanta, 58 (3): 543-550 (2003);

Direct physical attachment methods vary but most designs utilize tubing (e.g. capillary) filled with indicating reagent. In some cases, the substrate is directly bound (e.g. epoxy) to the fiber surface [Ming-Ren, S. Fuh, L. W. Burgess, T. Hirschfeld, G. D. Christian and F. Wang, The Analyst, “Single fiber optic fluorescence pH probe”, 112 (8), 1159-1163 (1987)]. Sensors of this type are typically fabricated in two principal steps. The steps involve immobilizing the indicator chemistry on a solid support material, and subsequently attaching this to the fiber. This method gives better reproducibility and is widely used. Background information for such a method can be found in, “Fiber-optic Chemical Sensors and Biosensors”, by O. S. Wolfbeis, Ed., (CRC press, Boca Raton, Fla., 1991) vol. 1. However, sensors fabricating in such a manner are limited to single analyte measurements. Dip coating methods are commonly used in many sol-gel sensor preparations and typically produce micron-thick sensing membranes per dip, with the resulting membrane(s) covering the entire surface of the fiber. Unlike direct physical attachment methods, the sensing layer can be produced in one step since the fiber tip is dipped in a formulation containing both the indicator chemistry and the solid support chemistry. Multiple coatings of different indicating chemistries can be sequentially added to the same fiber, producing multianalyte sensors. Background information for such coatings can be found in “Multilayer sol-gel membranes for optical sensing applications: single layer pH and dual layer CO₂ and NH₃ sensors,” by D. A. Nivens, M. V. Schiza, and S. M. Angel, Talanta, 58 (3): 543-550 (2003); and in “Use of a 2D to 1D dimension reduction fiber-optic array for multiwavelength imaging sensors,” by M. V. Schiza, M. P. Nelson, M. L. Myrick and S. M. Angel, Appl. Spectrosc., 55 (2), 217-226 (2001).

Such multianalyte sensor designs can suffer from issues of chemical compatibility and cross sensitivity. Sensors fabricated by dip coating have not been shown to be reproducible and do not offer spatial discrimination of the individual sensing layers, since each target analyte must interact with the indicator chemistry of a particular layer and produce an optically distinct signal (e.g. fluorescence or absorption).

Photopolymerization methods are among the earliest methods used for fiber-based sensor fabrication. In recent years, Walt et al (U.S. Pat. Nos. 5,244,636; 5,250,264 and 5,320,814 to David R. Walt and Steven M. Barnard, assigned to Trustees of Tufts College, patented Jun. 14, 1994, describe fiber optic sensors used for detecting at least one analyte of interest in a fluid sample) advanced this method by demonstrating that unique patterns of indicator chemistries can be covalently attached directly to the tips of optical fiber bundles, comprised of thousands of densely packed fibers [S. M. Barnard and D. R. Walt, “A fiberoptic chemical sensor with discrete sensing sites”, Nature, 353 (6342) 338-340 (1991). B. G. Healy, S. E. Foran, D. R. Walt, Science, 269, 1078 (1995)].

Specifically, such polymerized arrays of indicator chemistries are typically produced by immersing the polished surface of the optical fiber tip in a polymerizable indicator chemistry and selectively “growing” the indicator chemistries on the ends of the optical fiber strands via ultraviolet radiation photopolymerization. Such sensor arrays are spatially discriminated using simple imaging techniques. Multianalyte sensors have been fabricated by immersing the fiber tip sequentially in different polymerizable solutions followed by photopolymerization. However, the order in which the sensing elements are added to the fiber surface is very important because of cross sensitivity issues [J. A. Ferguson, B. G. Healy, K. S. Bronk, S. M. Barnard and D. R. Walt, “Simultaneous monitoring of pH, CO₂, and O₂ using an optical imaging fiber.” Anal. Chim. Acta 340, 123-131 (1997)]. Furthermore, such arrays are non-uniform, resulting from the lack of control during the photopolymerization step. This leads to sensors that are not reproducible in their response.

### SUMMARY OF THE INVENTION

In the present invention, rigid pin printing tool technology is utilized to apply one or more indicator chemistries on an optical array. Each indicator chemistry can contain one or more light energy absorbing dye(s) whose optical characteristics change in response to a target ligand or analyte of interest. By spectrally monitoring such changes using fluorescence and/or absorption spectroscopy, detection and/or quantitation of the target ligand or analyte is obtained. One or more ligand-specific indicator chemistries are contact printed using rigid pin technology in a known software automated pattern. Simultaneous detection and/or measurement of such ligands or analytes are accomplished using optical imaging techniques to spatially register each microdot.

In particular, the present invention is directed to a method of producing a chemical sensor that includes: providing an optical array; and contact printing one or more indicator chemistries to the optical array using one or more rigid pin printing tools, wherein the indicator chemistries can optically change due to a detected ligand or analyte of interest.

Another aspect of the present invention is directed to a chemical sensor production system capable of producing chemical sensors having one or more contact printed indicator chemistries arranged in predetermined patterns; wherein each such indicator chemistries can optically change due to a detected ligand or analyte of interest.

A further aspect of the present invention is directed to a chemical sensor that includes one or more contact printed indicator chemistries on an optical array; wherein the printed indicator chemistries can optically change due to a detected ligand or analyte of interest.

Accordingly, the present invention provides chemical sensors and a chemical sensor production system and method for producing such chemical sensors using rigid pin printing tool technology. Such produced sensors have applications in the biomedical, environmental, occupational safety, process control, and biowarfare fields.

### BRIEF DESCRIPTION OF THE DRAWINGS

FIG. 1 is a beneficial configuration of a printing station for printing patterns of microdots onto optical arrays.

FIG. 2 illustrates imaging in real-time, a contact-printed a microdot.

FIGS. 3(a)-3(d) illustrate example software automated contact-printed microdot configurations.

FIG. 4 shows a bright-field image of a 6-around-1 applied microdot configuration capable of a single analyte measurement.

FIG. 5 shows a bright-field image of a 6-around-1 applied microdot configuration capable of a multi-analyte measurement.

### DETAILED DESCRIPTION OF THE INVENTION

Referring now to the following detailed information, and to incorporated materials; a detailed description of the invention, including specific embodiments, is presented.

Unless otherwise indicated, numbers expressing quantities of ingredients, constituents, reaction conditions and so forth used in the specification and claims are to be understood as being modified by the term “about.” Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical value, however, inherently contains certain errors necessarily resulting from the standard deviation found in their respective testing measurements.

General Description

The present invention provides a contact-based rigid pin tool system/method for applying “microdots” of indicator chemistries to optical arrays to produce one or more analyte chemical sensors that can detect and/or monitor target ligands or analytes of interest. The present invention provides an improvement over a similar system that incorporates microjet technology to dispense indicator chemistries as utilized herein, (See Incorporated by reference, Co-pending U.S. application Ser. No. 09/709,047, titled: “Chemical Sensor System Utilizing Microjet Technology” by Brown et al. for more detail).

A particular beneficial feature of the present invention, as disclosed herein, is the use of solid rigid pin tools, such as, but not limited to, stamp pins, pins having concave bottoms, and pins arranged with slots, wherein such slots, concave features or flat surfaces operate as a reservoir for indicator chemistry sample loading and spotting with the capability of eliminating cross contamination issues through appropriate cleanings procedures or through multiple pin use. Such pin(s) can be mounted in a print head arranged in a commercial and/or custom printing platform such that the pins float under their own weight when contacted with a desired surface site. The printing platform is arranged to hold the surface to be contacted (e.g., an optical array) and the indicator chemistries are applied in a predetermined pattern automatically by custom and/or commercial software.

In the printing process, the one or more pins (directed via software) are dipped into the indicator solution (often disposed in solution wells), which results in the transfer of a small volume of indicator chemistry solution of less than about 1.0 μl onto the tips of such pins. By contacting “touching” such pins as utilized herein, onto a functionalized surface of optical arrays or disposable sheaths arranged on such arrays, the volume of indicator chemistry material, held by the pins, is applied in a spot having a diameter of often less than about 500 microns, which is determined by the surface energies of the pin, the optical surface itself, and the surface tension of the indicator chemistry.

Accordingly, by contact printing predetermined indicator chemistries onto desired surfaces using a software automated system, chemical sensors that include indicator chemistries whose optical characteristics change in response to the target ligand or analyte can be economically and efficiently manufactured.

Specific Description

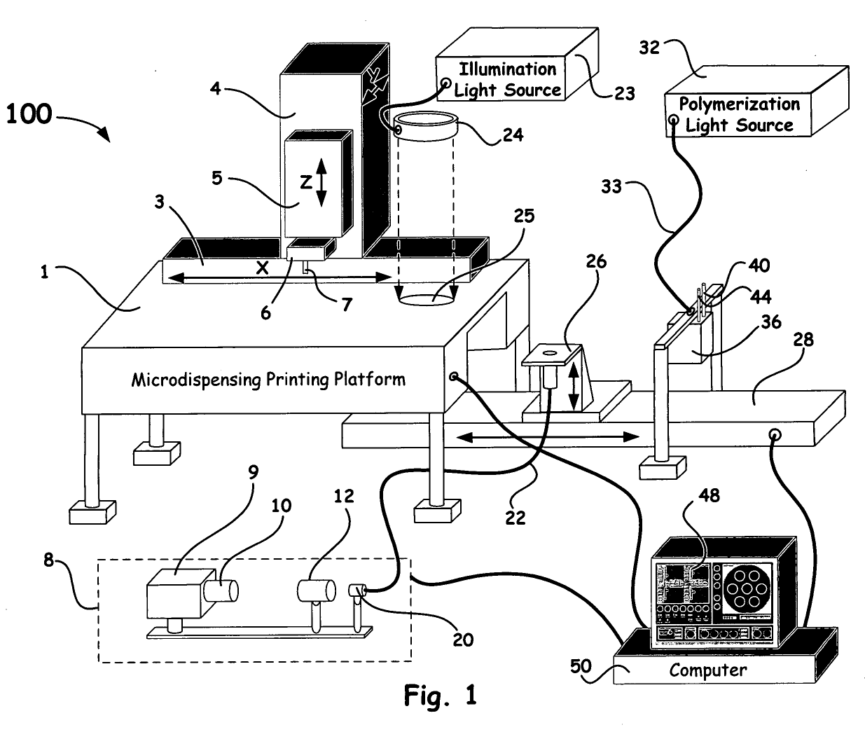

Turning now to the drawings, FIG. 1 shows an example basic beneficial arrangement of a microdispensing printing station, generally designated by reference numeral 100, utilized in printing patterns of chemically configured microdots onto optical arrays, such as, the tips of optical fiber bundles. More specifically, the system shown in FIG. 1 utilizes a non-capillary, rigid pin printing tool contact-based technology, to provide pluralities of indicator chemistries on predetermined optical arrays, such as, but not limited to fused fiber optic tapers, coherent capillary arrays, image conduits, clad rods, optical fiber bundles, etc.

Indicator chemistries can include, but are not limited to, one or more light energy absorbing dye(s) whose optical characteristics change in response to a target ligand or analyte. Optical imaging techniques can provide spatial registering for each microdot. Such indicator chemistries, printed as patterns of microdots on optical arrays, can be utilized as chemical sensors to provide qualitative and/or quantitation detection of a target ligand or analyte of interest by incorporating optical techniques, such as fluorescence and/or absorption spectroscopy.

Generally, station 100 can include, but is not limited to, a contact-based microdispensing printing platform 1; and an imaging vision system 8 (shown enclosed in a dashed box) having, for example, an imaging device 9, such as, for example a pixilated imaging device, often a charge coupled device (CCD) and/or any imaging device constructed to the design output parameters for system 100, coupled to one or more optical filtering or refractive components, such as, for example, a lens 10 and a collimating optic 12. A fixture 20 can be provided for holding the proximal end of an optical fiber image guide (e.g. an optical fiber bundle 22).

In addition, system 100 can include an illumination source 23, such as, optical coherent sources or filament sources, to be directed by an optical illumination means, such as, a refractive or reflective optic, often a ring light illuminator 24 for uniform illumination (as shown with dashed directional arrows in FIG. 1) of an optical array, such as, optical fiber bundle 22, while printing one or more patterns of microdots onto the tip of optical fiber bundle 22. Such an arrangement allows images to be acquired by imaging vision system 8 during printing, as described herein by the present invention, for real-time inspection and quality control purposes.

A vertical translating platform 26 (as shown in FIG. 1 with directional double arrows) capable of securing optical fiber bundle 22 and a linear horizontal translation stage 28 (also shown in FIG. 1 with directional double arrows) are configured to translate optical fiber bundle 22 into position at a predetermined site 25 within microdispensing printing platform 1 for printing the predetermined patterns of microdots at the tip of optical fiber bundle 22.

In addition, linear horizontal translating stage 28 and vertical translating platform 26 holding, for example, optical fiber bundle 22, is arranged with auto-motion control (e.g., via a graphical computer interface software program) to position the tip of optical fiber bundle 22 under a photo-polymerization chamber 36 for processing as detailed below.

An electromagnetic source 32 can be configured to direct predetermined wavelengths from greater than about the ultraviolet wavelengths (e.g., greater than 190 nm) along a conduit, such as, an optical fiber guide, more often a liquid light guide 33, to photo-polymerization chamber 36. Photo-polymerization chamber 36 is configured to house the distal portion of liquid light guide 33 in addition to housing a probe 40 for monitoring the humidity and an inlet 44 for purging photo-polymerization chamber 36 with a gas, such as, humidified N₂ gas.

The general concepts have been described above for FIG. 1. Specifically, an optical array, such as, but not limited to, optical fiber bundle 22, is loaded onto platform 26. A computer 50 having custom and/or commercial software (e.g., a graphical interface control means 48) can direct platform 26 to predetermined site 25 within contact-based microdispensing printing platform 1. A print head 6, which contains one or more rigid pin printing tools 7 of the present invention, is then directed via graphical interface control means 48 to a homing position via a robotic positioner i.e., X, Y, Z translation stages 3, 4, 5 (shown with accompanying directional arrows) before picking up a sample (i.e., an indicator chemistry). Print head 6 is then positioned via software directed X, Y, Z translation stages 3, 4, 5 above fiber bundle 22 but not touching fiber bundle 22.

Once positioned, one or more pins 7, which float under their own weight, can be software enabled to print “spot” one or more microdots, each having a predetermined indicator chemistry, onto the fiber bundle 22 surface or an optically coupled surface, such as a disposable protective sheath, at single site or in a predetermined pattern of sites. The printing process is viewed in real-time via imaging vision system 8.

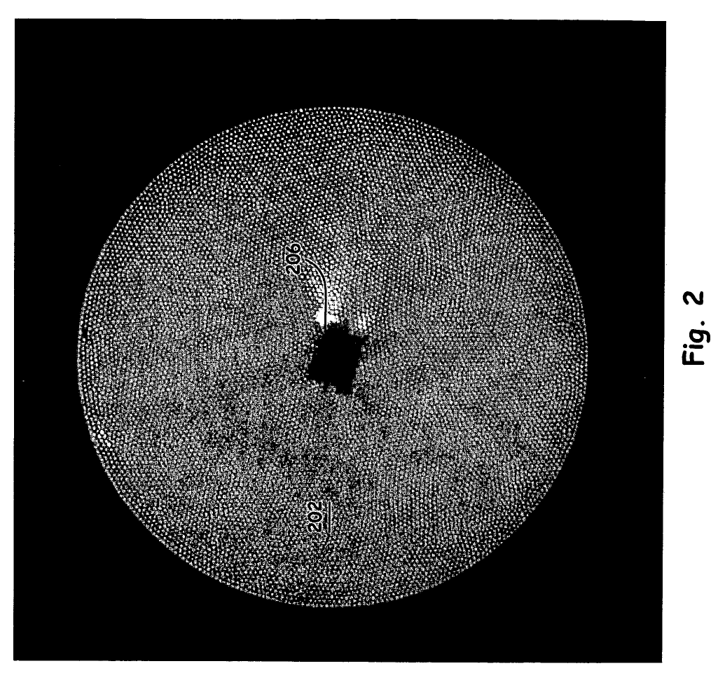

FIG. 2 illustrates an example technique for positioning a rigid pin printing tool 206, such as, for example a stamp tool, as shown in FIG. 2, so as to provide a reference coordinate for subsequently applied microdots on an optical array, such as, for example optical fiber bundle 202. First, rigid pin printing tool 206 is centered above fiber bundle 202, then can be enabled to contact the surface to set a predetermined position (i.e., reference coordinate). As another arrangement, rigid pin printing tool 206 can be brought into proximate contact with a surface (e.g., less than about 100 microns) to provide a shadow image of a rigid pin tool for purposes of alignment. As rigid pin printing tool 206 is brought into contact or substantially in contact with the surface of fiber bundle 202, an image of the distal portion of rigid pin printing tool 206 is captured in real time via vision imaging system 8, as shown FIG. 1. Such an arrangement allows a user to set an x:0, y:0, z:0 printing position on the fiber bundle 22 surface to enable a variety of print patterns, such as, for example, 6 microdots arranged about a substantially centered microdot.

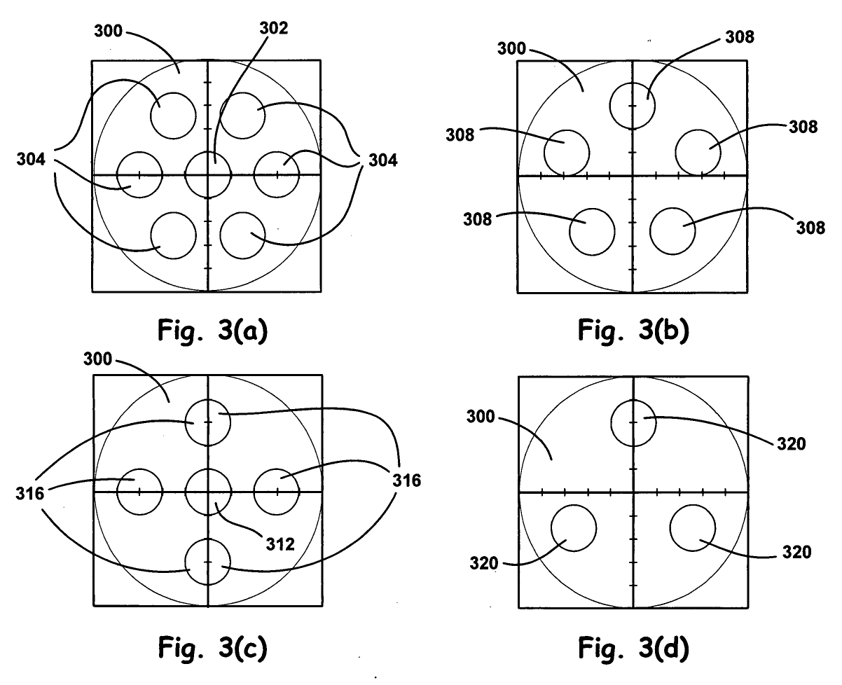

FIGS. 3(a)-3(d) illustrates, by way of example only, a variety of customized microdot print patterns capable of being applied by the present invention. FIG. 3(a) shows a 6-around-1 (6 microdots 304 around a centrally applied microdot 302) arranged pattern on the surface of, for example, a fiber bundle 300. FIG. 3(b) shows a five microdot 308 arranged pattern on the surface of, for example, fiber bundle 300. FIG. 3(c) shows a 4-around-1 (4 microdots 316 around a centrally applied microdot 312) arranged pattern on the surface of, for example, fiber bundle 300. FIG. 3(d) shows a three microdot 320 arranged pattern on the surface of, for example, fiber bundle 300.

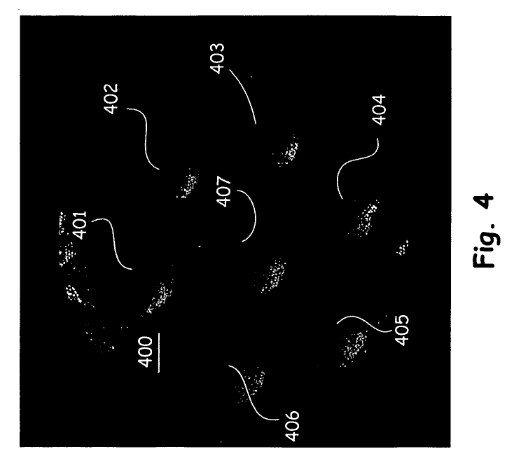

FIG. 4 illustrates a bright-field image (e.g., as imaged by imaging vision system 8, as shown in FIG. 1) of an example 6-around-1 pattern (6 microdots 401, 402, 403, 404, 405, 406) around a centrally applied microdot 407 as applied to an optical fiber bundle 400 having a diameter of about 500 microns. Each microdot is about 100 microns and in this example arrangement, each microdot includes a polymer formulation containing immobilized (pH sensitive) acryloylfluorescein dye.

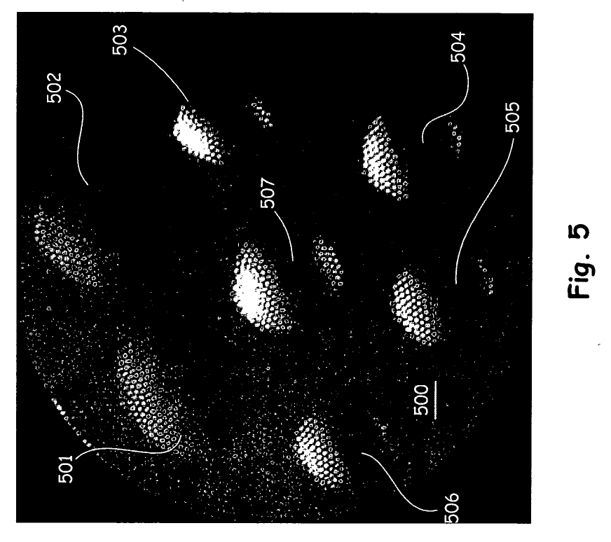

FIG. 5 shows a bright-field image of the polished surface of the distal end of an optical fiber image guide 500 onto which a 6-around-1 array of polymer immobilized indicator chemistries (i.e., multi-analytes) 501, 502, 503, 504, 505, 506, and 507 have been printed. Such polymer based micron-sized dots (i.e. microdots of multi-analytes) can be printed using contact-based microdispensing printing station 100, as illustrated in FIG. 1. The two largest microdots (i.e., 501 and 502) of similar size are acrylamide based hydrogels that include acryloyfluorescein indicator chemistry for sensing pH changes in solution. The remaining 5 microdots of similar size (i.e., 503, 504, 505, 506, and 507) are also acrylamide-based hydrogels. Of these 5, all but central microdot 507 contains a FRET-based polypeptide sequence indicator chemistry for detecting select enzymes. Central microdot 507 in particular, contains no indicator chemistry and serves as an experimental control. The acrylamide formulations for the pH and enzyme indicators are different formulations, which accounts for the size differences.

Control of the dimensions and aspect ratio of a printed microdot to a given specification is obtained by adjusting the following variables:
(a) the surface tension of the polymer formulation (e.g. controlled using surfactants)
(b) surface energy of the polished optical array surface (e.g. controlled using silanization method functionalization or low-wet coatings)

In addition to hardware, a control system software is utilized that can include, a graphical programming environment, such as, for example, LabVIEW. LabVIEW in particular, is specifically tailored to the development of instrument control applications and facilitates rapid user interface creation. A single user interface permits a user to manually position a rigid pin printing tool of the present invention, zero such a tool at the center of, for example, an optical fiber array, such as, but not limited to ICCD arrays, optical fiber bundles, etc., to create a custom printing pattern using a pattern editor, and execute an automated printing routine.

A user can create and visualize a customized pattern of microdots simply by using a drag-and-drop tool from a palette of up to conceivably 1596 color-coded chemistries. FIGS. 3(a)-(d), as shown above, illustrates such example customized arranged patterns of the present invention. Each chemistry is color-coded, as specified by the user, within the software and mapped to one well in a standard well plate. The user can save this pattern to a file, or load a previously saved pattern to the pattern editor. After placing dots on a pattern editor template, individual dots can be selected and the position finely tuned by adjusting coordinates. The order in which microdots are printed is determined by the placement order in the pattern editor. In multi-chemistry printing, all microdots of like chemistries are printed in sequence.

An automated routine executes a single printing cycle for each indicator chemistry specified in a desired custom pattern. The printing cycle includes chemistry pickup from a specified well in a well plate, conditioning the sample delivery of the rigid tool by printing a specified number of microdots on a predetermined blotting substrate (e.g., a glass slide), printing the desired microdot configuration on a predetermined optical array, such as, for example, optical fiber bundles, and cleaning the rigid pin printing tool according to a user specified wash cycle before the next chemistry pickup. In a settings menu, a user can specify which wells are used for sample pickup, the stages in a wash cycle, the conditioning procedure, the descent speed of the rigid tool during printing, and the amount of time the tool rests on the printing surface. It is possible to pause the automated routine, make modifications to the pattern or wash cycle, realign the rigid tool and optical array, or manually position the rigid tool before resuming the routine. Spectroscopic measurements can be made using, for example, an imaging spectrometer.

Returning to FIG. 1, once a predetermined pattern has been transferred to the surface of fiber bundle 22, fiber bundle 22 can be positioned via translation platforms 26 and 28 to a coordinate position under photo-polymerization chamber 36. From such a position, the printed microdots on an optical array surface can be exposed to predetermined optical wavelengths having a desired power density via light guide 33 for polymerization processing.

Photo-polymerization chamber 36 can be designed to have a port 40 to produce a humidified nitrogen gas-purged atmosphere and a probe 44 to monitor the relative humidity. For example, photo-polymerization chamber 36 can be beneficially arranged to have an 80% or higher relative humidity and acrylamide formulations that can include a bisacrylamide crosslinker and acryloylfluorescein. Moreover beneficial optical polymerization parameters within photo-polymerization chamber 36 can include an irradiance of about 500 mW/cm², an exposure time of about 45 sec, and a wavelength range between about 320 nm and about 500 nm. While photo-polymerization as described above is a beneficial embodiment of the present invention, other polymerization techniques, such as, but not limited to thermal techniques, chemical methods, ionization methods, plasma methods, and electro-initiation methods, etc., can also be employed in various arrangements with the disclosed arrangements herein without departing from the spirit and scope of the application.

Additional characteristics of the sensor system and associated apparatus include the following:

1. Indicators

One or more indicators of the present invention can be coupled to the surface of an optical array. Indicator chemistries, which can be contact printed to an optical array surface, includes such indicators and the medium (e.g., polymer matrix) to which it is immobilized (e.g., covalently, entrapped, etc.), wherein each indicator can include, for example, at least one light energy absorbing dye whose optical characteristics change in response to a target ligand or analyte of interest. Light absorbing dyes are typically divided into two different classes: fluorophores—those compositions that emit light energy after absorption; and chromophores—those compounds that absorb light energy and internally convert this energy to kinetic or heat energy. These dyes can, in addition, be linked to other materials such as enzyme peptide sequences and antibody conjugates that interact with the target ligand. Specific examples are provided below.

a. Chromophores

Some absorptive dyes are the family of triphenylmethanedyes, such as malachite green and phenolpthalein, and the family of monoazo dyes that include the mordant browns, oranges, yellows and reds.

b. Fluorophores

There are many fluorescent dyes used in chemical assays. The most common are the xnathine dyes (e.g., fluroescein and rhodamine), oxazine dyes (nile blue and cresyl violet), the coumarins, and the more recently developed bimanes. Direct measurement of pH, for example, can be made using fluorescent dyes.
c. Fluorescent Antibody Conjugates

Antibodies are proteins synthesized by an animal in response to a foreign substance, called an antigen. Antibodies have specific affinity for the antigens elicited by their synthesis, with the capability to discriminate differences of a single residue on the surface. Fluorescent antibody conjugates can therefore be used in a solid phase immunoassay to quantitate the amount of a protein or other antigen. These tests, currently referred to as enzyme-linked immunosorbent assays (ELISA), are fairly rapid and convenient. During an ELISA assay, an antibody is attached to a polymeric support and exposed to the target protein. After washing the support to remove any unbound molecules, a second antibody specific for a different site on the antigen is added. The amount of second antibody added to the support is proportional to the quantity of targeted antigen in the sample. This second antibody is also linked to an enzyme, such as alkaline phosphatase, that can rapidly convert a colorless substrate into a colored product, or a nonfluorescent substrate into a fluorescent product. The primary limitations of this technology are the multiple washing and steps necessary to reach a fluorescent product and the nonspecific binding that occurs with some antibody substrates. These limitations make creation of an in vivo device challenging. The benefit, however, of using ELISA assays is the relatively huge number of antibody-based tests already available for many target diseases (such as pregnancy, HIV, etc.).

d. Fluorescent Enzyme (FRET)-Based Peptide Sequences

Fluorescence resonance energy transfer (FRET) is a distance-dependent interaction between the electronic excited states of two dye molecules in which excitation is transferred from a donor molecule to an acceptor molecule without substantial emission photons. The efficiency of FRET is dependent on the inverse sixth power of the intermolecular separation, making it useful over distances comparable with the dimensions of biological macromolecules. Thus, FRET is an important technique for investigating a variety of biological phenomena that produce changes in molecular proximity.

The present invention utilizes enzyme (FRET)-based peptide sequences because of the high specificity in the catalyzed reactions and reactants. An enzyme can catalyze a single chemical reaction (such as cleaving a peptide chain) or a set of closely related reactions. The activity of such proteinases, i.e., enzymes, can be determined by the rate at which the enzyme cleaves a specific amide linkage that binds two amino acids of a particular sequence in the protein substrate. However, rather than determine the rate at which an intact protein is cleaved, sensitive assays of the present invention have been utilized, which use a short amino acid sequence that can be recognized by, for example, a collagenase. Such sequences are usually only six to ten amino acids long. Such a polypeptide is prepared with two different fluorescent dyes (rhodamine and fluorescein), one at each end of the substrate molecule. These dyes are specially chosen because they form an energy transfer (ET) pair, such that when the dye molecules are within a minimal distance from one another, energy absorbed by fluorescein (the donor) is transferred directly to the nearby rhodamine (the acceptor) and therefore can be monitored using FRET. The efficiency of the transfer process is dependent on several factors, but two important requirements are: (1) that there be overlap between the emission spectrum of fluorescein and the excitation spectrum of rhodamine, and (2) that the dye molecules be located within a limited distance of one another, generally less than about 100 nm. In the absence of enzyme activity, fluorescein absorbs blue light. However, rather than lose this energy as fluorescence, the energy is efficiently transferred to the nearby rhodamine attached just a few amino acids away on the short polypeptide. When the substrate molecule is subjected to collagenase activity, the molecule can be cleaved at a specific amino acid sequence between the two dyes of the ET pair. The fragments that result from this activity separate in solution substantially beyond the minimal distance allowed for energy transfer to occur. Consequently, the energy absorbed by fluorescein is not transferred to rhodamine but rather is emitted as fluorescence from fluorescein's emission manifold with a maximum at about 512 nm. The change in the ratio of light emitted from fluorescein (about 512 nm) and from rhodamine (about 564 nm) is a measure of enzyme activity. By incorporating FRET, such an approach can be used to measure the activity of metalloproteinases other than collagenase. Because each metalloproteinase enzyme recognizes a different substrate amino acid sequence, indicator chemistries can be developed that separately assay the activity of each of the targeted metalloproteinases. This can be particularly valuable for a wide range of diseases that activate an undesirable immune response. In particular, this method is beneficial for detection of periodontal disease activity, where measurement of a single biomarker is often inadequate to make an accurate diagnosis. Table 2 below lists fluorogenic probes that have been evaluated and used to measure the activity of several matrix metalloproteinases.

TABLE 2
Fluorogenic substrates for various MMPs.

| | | | | |
|---|---|---|---|---|
| MMP | Family | Specific Enzyme | Mwt (kDa) | Probe Sequence |

| | | | | |
|---|---|---|---|---|
| MMP-1 | collagenase | interstitial collagenase | 42 | Dnp-Pro-Leu-Ala-Leu-Trp-Ala-Arg-NH₂ |
| MMP-2 | gelatinase | gelatinase A | 72 | Mca-Arg-Pro-Lys-Pro-Tyr-Ala-Nva-Trp |
| | | | | Met-Lys(Dnp)-NH₂ |
| MMP-3 | stromelysin | stromelysin-1 | 45 | Dnp-Pro-Tyr-Ala-Tyr-Trp-Met-Arg-OH |
| MMP-7 | gelatinase | matrilysm | 19 | Mca-Pro-Leu-Gly-Leu-Dpa-Ala-Arg-NH₂ |
| MMP-8 | collagenase | PMN collagenase | 65 | Dnp-Pro-Leu-Ala-Tyr-Trp-Ala-Arg--NH₂ |
| MMP-9 | gelatinase | gelatinase B | 92 | Dnp-Pro-Leu-Gly-Met-Trp-Ser-Arg-NH₂ |

e. Optically Responsive Particles

Particles, in some embodiments, possess both the ability to bind an analyte of interest and to create a change in the optically detected signals. In general, these particles can be conveniently organized into three classes: polymer-based, inorganic crystals, and quantum dots. The first type of particle can include a polymeric material, such as polystyrene, acrylamide, dextrose, etc. These polymer beads can be optically encoded (e.g. with organic dyes) to provide unique signatures. In one embodiment several different bead sets can each be doped with different amounts of a single organic dye, allowing unique optical identification based solely on the strength of the detected fluoresecent signal. Further complexity can be added by doping these polymeric beads with combinations of optical dyes, where each dye has a given spectral emission. This method is commonly used in flow cytometry instruments to provide mobile sensing platforms. The polymer particle itself, the organic dye used to dope the particle, or the attached indicator chemistry (e.g. antibodies, oligopeptides, DNA, etc.) can all serve as the indicator chemistry that responds to the target analyte. One beneficial embodiment, for example, uses optically encoded microbeads with attached recognition antibodies that respond optically to an analyte of interest.

The second type of particle, optically active inorganic crystals, can also be used as sensors or sensor-containing platforms. One class of these crystals that has desirable characteristics for this type of application is upconverting phosphors. These compounds convert light of longer wavelengths into higher energy, lower wavelength phosphorescence. This is desirable since longer wavelength light sources, particularly diode based laser systems, are much more available and inexpensive than lower wavelength sources. It is possible to create multiple upconverting phosphors with distinct spectral characteristics, allowing unique identification of each crystal set. These can then behave in a similar fashion to organically dyed polymeric microbeads as sensors, or sensor-containing supports. They have the advantage, when compared to organic dyes, of being much more optically stable and less suspectible to light or temperature-based degradation (i.e. photobleaching).

The third class of chemically sensitive particles, quantum dots, are relatively new and have great promise as optical labels. These particles are typically 1-100 nm in size, composed of materials such as silicon, germanium arsenide, and other semiconductor-type materials. Quantum dots interact with light in a very different method than fluorescent-based dyes, with several advantages. While fluorescent emission typically has a relatively broad spectral bandwidth (20-60 nm), quantum dots in theory can have sub-nanometer type spectral bandwidths. This aspect makes them very attractive for spectral multiplexing schemes, where each quantum particle is easily identified by the wavelength of light it emits. In addition, quantum particles of the material but different size emit light at different wavelengths, but can all be excited at a single wavelength. This has very practical advantages when designing sensor instrumentation, since a single light source can be used to produce multiplexed signals. The spectral emission of quantum particles is very susceptible to surface effects. These effects can be used as a sensor medium, where interaction with different analytes of interest produce shifts in the spectral emissions of the particles, or the surface can be inactivated and the particles used as optically-active labels for other recognition moieties, such as antibodies, oligonucleotides, etc.

2. Polymer Matrix

When forming and depositing each indicator chemistry in a microdot, it is desirable to combine the absorbing dye with monomer formulations to create a polymerizable mixture. A variety of different polymerization processes are known, including thermal techniques, photo-initiated methods, chemical methods, ionization methods, plasma methods, and electro-initiation methods. The most commonly used methods in microdot applied processes use thermal and/or photo-initiated methods. There are several key characteristics a polymer formulation is designed to have if it is to be used in contact printing indicator chemistries. The polymer formulation selected requires the appropriate chemical and physical properties (such as polarity and viscosity) for forming small, evenly distributed microdots on a given optical substrate. In addition, the chosen polymer matrix allows intimate interaction with the target ligand maximizing sensor sensitivity and minimizing sensor response time. By selecting polymers that are wettable, only slightly cross-linked, and biologically compatible, it is possible to minimize the effects of substrate immobilization and maintain a solution-phase-like environment. There are several types of polymers to choose from which are compatible with enzymes, including polyacrylamides, polyhydroxyethylmethacrylate, and various phosphazene polymers.

3. Microdots

Microdots of the present invention are often micron sized (e.g., less than about 500 microns) but can be nano-sized particles (e.g., about 100 nanometers) of polymer spots that can, but not necessarily are required to, contain an indicator as disclosed herein. Such microdots can also be arranged to include additional layers (i.e., one or more layers) of either a polymer membrane (e.g., a hydrophobic membrane applied to a polymerized microdot that includes an indicator immobilized in a hydrophilic membrane) and/or an indicator immobilized in a polymer (i.e., an indicator chemistry) applied to a polymerized spot. Such an example embodiment in the former case can be a sensor utilized as, for example, a gas sensor. A latter example can include an enzyme immobilized in a membrane with an accompanying indicator in a membrane.

4. Optical Array Substrate

The optical array substrate on which the indicator micodots are placed requires several basic properties. The surface of such a substrate must be accessible to incident and emitted light energy. In the case of a transmission based measurement, the substrate includes a transparent media such as glass, ceramics, and some plastics. A transparent substrate is not necessary, however, for a reflection based measurement, since the indicator microdots can be accessed from either side of an optical array substrate. The surface of the substrate is designed to permit minimal spreading of the microdots during the printing process while still maintaining good adhesion between the microdot and the optical substrate. Some type of surface preparation (i.e., functionalizing), such as glass silanization, may be necessary to make this feasible.

Two types of measurements are generally made: in vivo, where the measurement is made directly in the sample volume; and in vitro, where a sample volume is collected and then exposed to a sensing apparatus as disclosed herein.

a. In Vivo Applications

For in vivo applications it is desirable to have the sensor portion contained in a probe capable of accessing the desired sample. The sensor, for example, can be incorporated in a mechanical periodontal probe for sampling the gingival crevicular fluid and saliva; a needle for accessing tissue; a catheter, endoscope, or guidewire for monitoring blood constituents; a cone penetrometer for making soil gas measurements; or a down well sampler for groundwater monitoring, among others. A fiber optic bundle is a natural choice for these applications, since fibers can guide light long distances with minimal loss of intensity and are very compact. An optical array, such as a standard fiber imaging bundle, may contain 1000's of individually clad optical fibers in a small diameter bundle (<500 μm). Since each microdot overlays at least one imaging fiber the orientation (i.e. rotation) of the bundle tip relative to the rigid tool printing element becomes less important, making sensor manufacture much easier and allowing many more indicator microdots to be placed in a given area. The microdots can either be printed directly on the distal end of the fiber bundle or printed on the tip of a disposable sleeve (e.g. plastic) that can be slipped over the end of the imaging fiber bundle.

b. In Vitro Applications

Although the physical constraints on in vitro sensor design are less than for in vivo probes, it is still desirable to analyze only a small sample volume at one time. There are several reasons for this. First of all, a small volume implies precise sampling from a specific location. This is important for measuring changes that may only occur in a very localized region. For example, periodontal disease activity can vary significantly in a single patient depending on what part of the oral cavity is probed. As a second example, groundwater and soil contamination can vary significantly in a small region, depending in part on the solubility of the contaminant and the geology of the surrounding area. Secondly, it is often difficult to obtain a large sample volume. This is particularly true of biomedical applications (such as blood glucose monitoring) or biowarfare scenarios where, even with preconcentration schemes, only very small quantities of the targeted agent are present in the air or groundwater. Finally, a small sample volume allows multiple measurements to be made at the same site without significant risk of sample dilution.

5. Contact Microdispenser Printing

The method provides a means of precisely printing many different materials in a given pattern and a wide variety of microdot geometries. By incorporating a printing platform that can direct rigid pin printing tools, microdots of predetermined indicator chemistries, such as indicator polymeric material, may be deposited onto optical array substrates, such as, but not limited to, the tips of optical fiber bundles, to form arbitrary patterns of arbitrarily sized optical elements.

6. Illumination and Detection of the Sensing Site

Electromagnetic energy, typically optical, is transmitted to a sensing site to detect optical changes in the indicator chemistry. The simplest types of optical sources include light emitting diodes (LEDs), lasers, laser diodes, and filament lamps, such as broad-band light sources. Such sources can be used in conjunction with optical filters, diffraction gratings, prisms, and other optical components to provide a specified spectral bandwidth of energy, often in the optical regime. Alternative forms of radiation such as bioluminescence, phosphorescence, and others can also potentially be employed. Although typical fluorophores require excitation wavelengths in the visible portion of the spectrum (300-700 nm wavelength), other wavelengths in the infrared and ultraviolet portion of the spectrum can also prove beneficial for illuminating the indicator chemistry(s). The transmitted, reflected, or re-emitted light from the sensing region can then be propagated to an optical apparatus for detection and/or some type of spectral and spatial filtering.

a. Spectral Filtering

The same techniques as those described above (i.e. optical filters, diffraction gratings, etc.) can be used to spectrally process changes in the light returning from the sensing region. There are several ways this spectral information available from each illuminated indicator microdot can be used. First, it can be used to register the spatial position of the specific indicator chemistry. A very simple approach, for example, would be to design one indicator microdot to emit blue light in the presence of a particular biomarker and to design a second indicator chemistry that emits green light in the presence of a different biomarker. The intensity of the emission from each microdot can then be correlated to the concentration of their respective targeted biomarkers. It may also be desirable to use fluorescently labeled microbeads, each with a unique spectral signature, with specific indicator chemistries attached. The limitation of using spectral filtering for registration purposes is the potential overlap that can occur between multiple emission wavelength bands. In addition, if multiple biomarkers are targeted, each can require its own specific dye with a corresponding spectral processing scheme and possibly different excitation wavelength. A simpler approach for registration of each indicator microdot is to use their spatial location on the optical substrate, as described below. The second and more practical use of spectral filtering is to separate the desired component of the emitted light from the incident radiation. In the case of fluorescence, this amounts to separating the incident excitation band from the transmitted or reflected emission band. This method is also intended to incorporate more complex spectral processing schemes of single and multiple dye conjugates, including multivariate analysis, ratioing, and other standard spectroscopic techniques.

b. Detection and Spatial Processing

The spectrally filtered light from the sensing region can be detected using photosensitive detectors such as photodiodes or photomultiplier tubes. Spatial filtering of the light is also possible with two dimensional detectors such as charge coupling device cameras (CCDs) and video cameras. The use of a two dimensional detection system allows direct registration of multiple indicator microdots, eliminating the need to use spectrally diverse absorbing dyes and their associated spectral filtering components. This greatly simplifies the optical apparatus necessary to measure changes in the indicator chemistry(s). If the geometry of the microdot pattern is axis symmetrical (such as the six-around-one pattern as shown in FIG. 4), it is necessary to include (or exclude) a “reference” microdot to determine the positions of the other indicator chemistries (other than the central microdot). In other cases, the different sizes of microdots having different chemistries can be used to register adjacent dots. For example, the largest microdots, as discussed and as shown in FIG. 5, are pH sensing microdots.

These detection schemes may or may not be coupled to fiber optic/fiber optic bundles depending on the need to remotely access the sensing sites. The data from the selected detector system can then be acquired, processed, and displayed to the user using available data acquisition/processing systems. Depending on the application, such systems can range from a very simple detection scheme where a positive identification lights an LED to much more complicated systems using a computer interface to process image information for simultaneous real-time monitoring of multiple constituents.

The system of the present invention has a wide range of uses. Examples of some of the uses are listed below to more fully illustrate the invention. There are additional uses of the present invention that are not described.

(1) Biomedical Applications—Biosensor systems constructed in accordance with the present invention can be used as biomarkers for infectious diseases, blood gas levels (O₂, CO₂, etc.), electrolyte concentrations (K⁺, Ca⁺, Li⁺, etc.), periodontal disease (metalloproteinases), polymerase chain reaction (PCR) products, and other clinically important parameters (pH, glucose, etc.).

(2) Environmental Applications—Chemical sensor systems constructed in accordance with the present invention can be used for monitoring hazardous materials such as heavy metal, hydrocarbons, and chlorinated hydrocarbons in both the groundwater and soil of contaminated sites.

(3) Occupational Safety—Chemical sensor systems constructed in accordance with the present invention can be used for making accurate dosimetry measurements of hazardous materials, such as carcinogens or mutagens present in hostile or potentially hostile environments. These can include compounds that are traditionally detected using flame ionization detectors (FID) or portable gas chromatographs.

(4) Process Control—Sensors systems constructed in accordance with the present invention can be implemented in assembly line type configurations for quality and process control type applications. Examples include measurements of gases emitted from fruits and vegetables and detection of contaminants in soft drink or bottled water solutions.

(5) Chem/Biowarfare Applications—Sensors systems constructed in accordance with the present invention can be developed for detection/early warning of airborne or water-based chemical and biowarfare agents such as anthrax.

Changes and modifications in the specifically described embodiments can be carried out without departing from the scope of the invention, which is intended to be limited by the scope of the appended claims.

1. A method of producing a chemical sensor, comprising:
providing an optical array; and
contact printing one or more indicator chemistries to said optical array using one or more rigid pin printing tools, wherein said one or more indicator chemistries can optically change due to a detected ligand or analyte of interest.

2. The method of claim 1, wherein said one or more rigid pin printing tools comprise solid pins configured with a concave bottom.

3. The method of claim 1, wherein said one or more rigid pin printing tools comprise solid pins configured with a flat bottom.

4. The method of claim 1, wherein said one or more rigid pin printing tools comprise solid pins configured with a slot.

5. The method of claim 1, wherein said optical changes due to a detected ligand or analyte of interest comprises in-vivo monitoring.

6. The method of claim 1, wherein said optical changes due to a detected ligand or analyte of interest comprises in-vitro monitoring.

7. The method of claim 1, wherein said ligand or analyte of interest is disposed within a fluid medium.

8. The method of claim 7, wherein said fluid medium comprises a liquid medium.

9. The method of claim 7, wherein said fluid medium comprises an airborne medium.

10. The method of claim 1, wherein said one or more indicator chemistries comprise one or more light absorbing dyes.

11. The method of claim 10, wherein said one or more indicator chemistries further comprise enzyme (FRET)-based peptide sequences.

12. The method of claim 10, wherein said one or more indicator chemistries further comprise enzyme antibody conjugates.

13. The method of claim 1, wherein said indicator chemistries further comprise an optically responsive particle.

14. The method of claim 13, wherein said optically responsive particle comprises at least one particle selected from: a quantum dot, a polymeric material, and an optically active inorganic crystal.

15. The method of claim 1, wherein said optical array comprises a bundle containing a plurality of fiber optic strands and wherein said step of printing one or more indicator chemistries comprises printing one or more indicator chemistries on the tip of said bundle of fiber optic strands.

16. The method of claim 1, wherein said optical array comprises at least one array selected from: fused fiber optic tapers, coherent capillary arrays, image conduits, clad rods, and optical fiber bundles.

17. The method of claim 1, wherein a protective sheath is adapted on the surface of said optical array for receiving said one or more indicator chemistries.

18. The method of claim 1, wherein said method further comprises polymerizing said printed said one or more indicator chemistries.

19. The method of claim 1, wherein said polymerizing step comprises at least one polymerization technique selected from: photo-initiation, thermal-initiation, chemical-initiation, ionization-initiation, plasma-initiation, and electro-initiation.

20. The method of claim 1, wherein an arranged printing pattern of said one or more indicator chemistries are predetermined via custom and/or commercial software.

21. The method of claim 1, wherein each of said one or more indicator chemistries can be configured as a polymerized microdot that is capable of being further configured with one or more additional layers of applied indicator chemistries or polymer matrix.

22. The method of claim 1, further comprising functionalizing the surface of said optical array for adhering said one or more indicator chemistries.

23. The method of claim 1, wherein said one or more indicator chemistries comprise multianalytes.

24. A chemical sensor production system, comprising:
a printing platform;
an optical array capable of being disposed within said printing platform;
one or more rigid pin printing tools adapted with said printing platform for contact printing one or more indicator chemistries on said optical array; wherein said indicator chemistries can optically change due to a detected ligand or analyte of interest; and
a polymerization chamber arranged to polymerize said printed one or more indicator chemistries.

25. The system of claim 24, wherein said one or more rigid pin printing tools comprise solid pins configured with a concave bottom.

26. The system of claim 24, wherein said one or more rigid pin printing tools comprise solid pins configured with a flat bottom.

27. The system of claim 24, wherein said one or more rigid pin printing tools comprise solid pins configured with a slot.

28. The system of claim 24, wherein said optical changes due to a detected ligand or analyte of interest comprises in-vivo monitoring.

29. The system of claim 24, wherein said optical changes due to a detected ligand or analyte of interest comprises in-vitro monitoring.

30. The system of claim 24, wherein said ligand or analyte of interest is disposed within a fluid medium.

31. The system of claim 30, wherein said fluid medium comprises a liquid medium.

32. The system of claim 30, wherein said fluid medium comprises an airborne medium.

33. The system of claim 24, wherein said one or more indicator chemistries comprise one or more light absorbing dyes.

34. The system of claim 33, wherein said one or more indicator chemistries further comprise enzyme (FRET)-based peptide sequences.

35. The system of claim 33, wherein said one or more indicator chemistries further comprise enzyme antibody conjugates.

36. The system of claim 24, wherein said indicator chemistries further comprise an optically responsive particle.

37. The system of claim 36, wherein said optically responsive particle comprises at least one particle selected from: a quantum dot, a polymeric material, and an optically active inorganic crystal.

38. The system of claim 24, wherein said optical array comprises a bundle containing a plurality of fiber optic strands.

39. The system of claim 24, wherein said optical array comprises at least one array selected from: fused fiber optic tapers, coherent capillary arrays, image conduits, clad rods, and optical fiber bundles.

40. The system of claim 24, wherein a protective sheath is adapted on the surface of said optical array for receiving said one or more indicator chemistries.

41. The system of claim 24, wherein an arranged printing pattern of said one or more indicator chemistries are predetermined via custom and/or commercial software.

42. The system of claim 24, wherein each of said one or more indicator chemistries can be configured as a polymerized microdot that is capable of being further configured with one or more additional layers of applied indicator chemistries or polymer matrix.

43. The system of claim 24, wherein the surface of said optical array is functionalized so as to adhere said indicator chemistries.

44. The system of claim 24, wherein said one or more indicator chemistries comprise analytes.

45. A chemical sensor, comprising:
an optical array;
one or more contact-printed indicator chemistries arranged on said optical array; wherein said indicator chemistries can optically change due to a detected ligand or analyte of interest.

46. The sensor of claim 45, wherein said indicator chemistries are capable of being contact printed with a rigid printing pin tool configured with a concave bottom.

47. The sensor of claim 45, wherein said indicator chemistries are capable of being contact printed with a rigid pin printing tool configured with a slot.

48. The sensor of claim 45, wherein said indicator chemistries are capable of being contact printed with a rigid pin printing tool configured with a flat bottom.

49. The sensor of claim 45, wherein said optical changes due to a detected ligand or analyte of interest comprises in-vivo monitoring.

50. The sensor of claim 45, wherein said optical changes due to a detected ligand or analyte of interest comprises in-vitro monitoring.

51. The sensor of claim 45, wherein said ligand or analyte of interest is disposed within a fluid medium.

52. The sensor of claim 51, wherein said fluid medium comprises a liquid medium.

53. The sensor of claim 51, wherein said fluid medium comprises an airborne medium.

54. The sensor of claim 43, wherein said one or more indicator chemistries comprise one or more light absorbing dyes.

55. The sensor of claim 54, wherein said one or more contact printed indicator chemistries further comprise enzyme (FRET)-based peptide sequences.

56. The sensor of claim 54, wherein said one or more contact printed indicator chemistries further comprise enzyme antibody conjugates.

57. The sensor of claim 45, wherein said indicator chemistries further comprise an optically responsive particle.

58. The sensor of claim 57, wherein said optically responsive particle comprises at least one particle selected from: a quantum dot, a polymeric material, and an optically active inorganic crystal.

59. The sensor of claim 45, wherein said optical array comprises a bundle containing a plurality of fiber optic strands.

60. The sensor of claim 45, wherein said optical array comprises at least one array selected from: fused fiber optic tapers, coherent capillary arrays, image conduits, clad rods, and optical fiber bundles.

61. The sensor of claim 45, wherein each of said one or more contact-printed indicator chemistries can be configured as a polymerized microdot that is capable of being further configured with one or more additional layers of applied indicator chemistries or polymer matrix.

62. The sensor of claim 45, wherein said one or more contact-printed indicator chemistries comprise multi-analytes.
